(19) United States
(12) Patent Application Publication
(10) Pub. No.: US 2007/0037205 A1
(43) Pub. Date: Feb. 15, 2007

(54) Methods and apparatus for cell based screening

(76) Inventors: Michael A. Palladino JR., Olivenhain, CA (US); Scott M. Glaser, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/585,565

(22) Filed: Oct. 24, 2006

Related U.S. Application Data
(63) Continuation of application No. 10/309,782, filed on Dec. 3, 2002.
(60) Provisional application No. 60/336,804, filed on Dec. 4, 2001.

(51) Int. Cl. C12Q 1/68; G01N 33/53; G01N 33/574; C40B 30/06

(52) U.S. Cl. 435/6; 435/7.1; 435/7.23

(57) ABSTRACT

Methods, kits and systems for identifying at least one compound, from a set of compounds, that modulates the growth or biological activity of a cell. Also high-throughput, tumor cell-based screening assay methods for identifying one or more cell-growth modulating compounds from among a library of compounds.

### CROSS REFERENCE TO RELATED APPLICATIONS

The present application is a continuation of, and claims priority under 35 U.S.C. § 120 from copending U.S. application Ser. No. 10/309,782 filed on Dec. 3, 2002 entitled “Methods and Apparatus for Cell Based Screening,” which application is incorporated by reference herein in its entirety and claims priority under 35 U.S.C. § 119(e) to U.S. Provisional Patent Application Serial No. 60/336,804, filed on Dec. 4, 2001, entitled “Methods and Apparatus for Cell Based Screening,” which application is incorporated by reference herein in its entirety.

### BACKGROUND OF THE INVENTION

1. Field of the Invention

The present invention relates generally to methods, systems, and kits for identifying novel and useful molecules and compounds from libraries, mixtures, extracts, and sets of molecules and compounds. Generally, the molecules and compounds modulate cellular growth and/or cellular activity.

2. Description of the Related Art

Numerous academic and industrial research efforts have been aimed at identifying novel target molecules that may exhibit therapeutic value or be drug targets. The pharmaceutical and biotechnological industries have recently been presented with a large number of new sources for potential target molecules, including proteomes, genomes, newly found organisms, libraries of these, and the like. However, in view of the large amounts of data and material with unrealized benefit, a challenge is to develop efficient and sensitive tools to assess the potential relevance of the numerous candidates and to rapidly select those relatively few candidates for which further development effort is justified.

In many settings, and particularly when working with complex mixtures or libraries of compounds, the concentration of an individual active compound may be sufficiently low that it would not be identified by currently available or otherwise conventional screening systems and methods. Similarly, the activity of an individual compound in a complex mixture or library of compounds may be sufficiently low that it would not be identified by currently available or otherwise conventional screening systems and methods. Therefore, conventional methods and systems are often incapable of identifying targets that are present in low levels or that have low activity, such as in complex mixtures and/or compound libraries.

There is, therefore, a need for screening systems and methods that provide for increased sensitivity. Such sensitivity may be sufficient to detect and identify target compounds that would be undetected by conventional systems and methods. Such needed systems and methods are desirable in the context of academic and industrial research efforts involved in the discovery of novel and beneficial compounds.

### SUMMARY OF THE INVENTION

One disclosed embodiment relates to a method of identifying at least one compound, from a set of compounds, that modulates the growth or biological activity of a cell. The method may include the steps of (a) contacting a cell to an agent that increases the sensitivity of the cell to cell death by at least three-fold, thereby yielding a sensitized or more differentiated cell; (b) contacting the sensitized cell to at least one compound from the set of compounds; and (c) identifying at least one compound that modulates the growth of the cell by detecting an indicia of cellular growth of the sensitized cell.

The agent, for example, may include one or more of the following: cytokines, interferons, growth factors, chemokines, chemotherapeutics, peptides, polypeptides, nucleic acid sensitizers, gene-based sensitizers, lipids, lipopeptides, sterols and their biosynthetic precursors, polysaccharides, lipopolysaccharides, phosphatase inhibitors, kinase inhibitors, temperature, pH, and the like. Examples of the cytokine may include IL2, IFN-γ, IL12, and TNF-beta (lymphotoxin), IL4, IL5, IL6, IL10, IL13, and the like.

The agent further may increase the sensitivity of the cell to cell death by a factor of at least about 4. The agent further may increase the sensitivity of the cell to cell death by a factor of at least about 5. The agent further may increase the sensitivity of the cell to cell death by a factor of at least about 6. The agent further may increase the sensitivity of the cell to cell death by a factor of at least about 9. The agent further may increase the sensitivity of the cell to cell death by a factor of at least about 10. The agent further may increase the sensitivity of the cell to cell death by a factor of at least about 12. The agent further may increase the sensitivity of the cell to cell death by a factor of at least about 15. The agent further may increase the sensitivity of the cell to cell death by a factor of at least about 20. The agent further may increase the sensitivity of the cell to cell death by a factor of at least about 30. The agent further may increase the sensitivity of the cell to cell death by a factor of at least about 50. The agent further may increase the sensitivity of the cell to cell death by a factor of at least about 100. The agent further may increase the sensitivity of the cell to cell death by a factor of at least about 500. The agent further may increase the sensitivity of the cell to cell death by a factor of at least about 1000.

Also, the set of compounds may be a complex mixture of compounds, and the like, for example. The method may be a high-throughput screen. The cell may be a tumor cell, and the like. Preferred examples of the cell include HT-29, LoVo, SW620 cell, mesothelioma cell lines, glioma cell lines, ovarian carcinoma cell lines, and human renal cell carcinoma cell lines. The cell may be infected by an intracellular parasite. The intracellular parasite may be a virus, a bacterium, a fungus, and a protozoa, and the like, for example. The cell may be an engineered cell or cell line, and the like.

The indicia of cellular growth may be detected by a technique that may include whole cell counting, viable dye staining, direct counting with a hemacytometer, monitoring DNA synthesis by ³H-thymidine incorporation and radiometric detection, BrdU incorporation, colorimetric/fluorescent detection with a labeled antibody reactive to BrdU, cell proliferation as monitored by crystal violet cellular staining and colorimetric detection, cell proliferation as monitored by sulforhodamine B cellular staining and colorimetric detection, and metabolic activity as monitored by MTT and calorimetric detection. The indicia of cellular growth may be, for example, metabolic activity in the sensitized cell, and the like. The indicia of cellular growth may be fluorescence of a dye in the presence of the sensitized cell, for example. The dye may be tetrazolium violet, 2-(p-Iodophenyl)-3-(p-nitrophenyl)-5-phenyl-tetrazolium chloride, resazurin, and the like, for example.

The biological and/or cellular activity may be antibiotic activity, anti-inflammatory activity, anti-cancer activity, CNS activity, cardiovascular and/or anti-angiogenic activity, renal activity, gastrointestinal activity, uterine activity, anti-parasitic activity, immunomodulatory activity, hematopoietic activity, metabolic activity, agonists, partial agonists, inverse agonists, reverse agonists, antagonists, competitive antagonists, non-competitive antagonists, and the like.

Another embodiment relates to a high-throughput, tumor cell-based screening assay method for identifying one or more cell-growth or differentiation modulating compounds from among a library of compounds. The method may include the improvement, which may include the step of contacting the tumor cells to an agent that increases the sensitivity of the tumor cell to cell death.

Examples of the agent may include cytokines, interferons, growth factors, chemokines, chemotherapeutics, peptides, polypeptides, nucleic acid sensitizers, gene-based sensitizers, lipids, lipopeptides, sterols and their biosynthetic precursors, polysaccharides, lipopolysaccharides, phosphatase inhibitors, kinase inhibitors, temperature, pH, and the like. The cytokine may be, for example, IL2, IFN-γ, IL12, and TNF-beta (lymphotoxin), IL4, IL5, IL6, IL10, IL13, and the like.

The agent may increase the sensitivity of the cell to cell death by a factor of at least about 3. The agent may increase the sensitivity of the cell to cell death by a factor of at least about 6. The agent may increase the sensitivity of the cell to cell death by a factor of at least about 10. The agent may increase the sensitivity of the cell to cell death by a factor of at least about 15. The agent may increase the sensitivity of the cell to cell death by a factor of at least about 30. The agent may increase the sensitivity of the cell to cell death by a factor of at least about 50. The agent may increase the sensitivity of the cell to cell death by a factor of at least about 100. The agent may increase the sensitivity of the cell to cell death by a factor of at least about 500. The agent may increase the sensitivity of the cell to cell death by a factor of at least about 1000.

The library of compounds may be derived from a natural source, for example. The library of compounds may be prepared by fractionating an extract, and the like, for example. The extract may be from a natural source, for example.

Another embodiment relates to a kit for identifying at least one compound, from a set of compounds, that modulates the growth or biological activity of a cell. The kit may include a container for contacting a compound or set of compounds with a sensitized cell and at least one additional component. The at least one additional component may be, for example, a sensitizing agent, a cell, a reagent for detecting an indicia of cellular growth and/or biological or cellular activity, a growth media, and the like for example. The container may be a well in a plate, a well or wells in a multi-well container, a test tube, and the like, for example.

### BRIEF DESCRIPTION OF THE DRAWINGS

FIG. 1 is a graphic representation of anti-tumor cell activities of 320 natural product extracts screened in the HT-29 cytotoxicity assay in the absence and presence of IFN-γ.

FIG. 2 is a graphic representation of the results of a cytotoxicity assay for HT-29 tumor cells (both with and without IFN-γ pretreatment) and NPS1518, a positive extract identified in a primary HT-29 screen.

FIG. 3 is a graphic representation of the results of a cytotoxicity assay for HT-29 tumor cells (both with and without IFN-γ pretreatment) and NPS1594, a positive extract identified in a primary HT-29 screen.

FIG. 4 is a graphic representation of the results of a cytotoxicity assay for HT-29 tumor cells (both with and without IFN-γ pretreatment) and NPS1386, a positive extract identified in a primary HT-29 screen.

FIG. 5 is a graphic representation of HPLC analyses of NPS1386 crude extract and partially purified NPS1386 compound.

FIG. 6 is a graphic representation of the results of a cytotoxicity assay for Jurkat human tumor cells and partially purified NPS1386 compound.

FIG. 7 is a graphic representation of the results of a cytotoxicity assay for HT-29 tumor cells (both with and without IFN-γ pretreatment) and a purified sample of the discovered compound designated 1583-05-AB.

### DETAILED DESCRIPTION OF THE PREFERRED EMBODIMENT

Generally, methods and systems are described herein for identifying potential drug and therapeutic candidates, and biologically active chemicals from complex mixtures of compounds or libraries. A method and system are described for identifying a compound that modulates cellular growth and/or the biological activity of a cell. In some embodiments the methods and systems may identify from complex mixtures, compounds that exhibit low activity or that are present in low concentration.

In one set of embodiments, a system and a method are provided for identifying at least one compound that modulates the growth or differentiation of a cell from a set of compounds. The method may include the steps of contacting a cell to an agent that increases the sensitivity of the cell to cell death or other cell growth events, thereby resulting in a sensitized cell. The sensitized cell may be contacted with at least one compound from the set of compounds. At least one compound that modulates the growth of the cell may be identified by detecting an indicia of cellular growth of the sensitized cell.

The invention described herein provides a major advance in screening systems and methods by providing increased sensitivity sufficient to detect and identify target molecules that have until now gone undetected. Thus, the present systems and methods provide significant benefits to academic research, industrial research, and other like efforts that are involved in the discovery of novel and beneficial molecules and/or compounds.

Conventional identification and screening methods lack sensitivity to find many potentially useful compounds. Many compounds may be present in small quantities and/or low concentration, rendering conventional techniques ineffective. Further, a compound may exhibit a low activity in a complex mixture so that it is not detected by conventional techniques. One benefit of the methods and systems disclosed herein relates to the ability to identify one or more compounds, that previously might have been undetected, by increasing the sensitivity of a cell to cell death such that sensitivity to a potentially advantageous compound is increased. In certain embodiments, the methods and systems can be used to identify oncology and inflammatory agents against tumor cells, for example.

In such embodiments, the cell may be any cell for which a compound is sought that modulates the growth of the cell. For example, the cell may be a tumor cell, a cell infected by an intracellular parasite (such as a virus, a bacterium, a fungus, a protozoa, and the like), or a cell infected by an extracellular microbe or pathogen (bacterium, protozoa, fungus, etc.). In preferred embodiments the cell may be a eukaryotic cell, more preferably a mammalian cell, and most preferred a human cell. In a preferred embodiment, an HT-29 human colon cell line is used. Additional cell lines may include, but are not limited to: human colon adenocarcinoma cell lines, LoVo, (Monti F, Lalli E, Bontadini A, Szymczuk S, Pini E, Tononi A, Fattori P P, Facchini A, Ravaioli A J Chemother 1994 October; 6(5):337-42), SW620, (O'Connell J, Bennett M W, Nally K, O'Sullivan G C, Collins J K, Shanahan F J Cell Physiol 2000 December; 185(3):331-8.), and the like; mesothelioma cell lines (Hand A, Pelin K, Mattson K, Linnainmaa K. Anticancer Drugs 1995 February; 6(1):77-82); glioma cell lines (Weller M, Malipiero U, Aguzzi A, Reed J C, Fontana A J Clin Invest 1995 June; 95(6):2633-43); ovarian carcinoma cell lines (Marth C, Helmberg M, Mayer I, Fuith L C, Daxenbichler G, Dapunt O. Anticancer Res 1989 March-April; 9(2):461-7); human renal cell carcinoma (RCC) cell lines (Rohde D, Hayn H K, Blatter J, Jakse G. Int J Oncol 1998 June; 12(6):1361-6). Chronic myelogenous leukemia cell lines (e.g. K562 cells) (Lee J T, Park S, Lee J H, Kim B K, Kim N K. J Korean Med Sci 1996 February; 11(1):26-32); and the like. Specific preferred examples, include, MCF-7 human breast carcinoma cells, NIH-OVCAR-3 ovarian carcinoma, HeLa human ovarian carcinoma, LNCaP human prostate carcinoma, CaCo-2 human colonic carcinoma cells, HT-29 cells and the like. In the case of a tumor cell, the methods and systems can be used to identify a compound that is effective at causing cell death or modulating cellular activity, for example. Further, the cell may be infected by a prion and the like. Also contemplated are engineered cells and cell lines. A cell line may be developed for use consistent with the present methods, systems and kits. For example, a cell line may be developed by means of genetic engineering or by selecting and isolating spontaneously occurring sensitive cells, or compound-induced sensitive cell lines. As a further example, a sensitive cell line may be engineered to impart to it IFN sensitivity, for example. The cell may be used in a screen or assay, for example, consistent with the methods, systems and kits described herein, to discover or identify compounds that modulate cell growth or activity, and the like.

The agent that increases the sensitivity of the cell can be one or more of the many agents that are known to those of skill in the art or any other agent that is subsequently discovered. The agent may be a single molecule, more than one molecule, a compound, and the like. Those of skill in the art are familiar with particular cells as described above, and agents that increase the sensitivity of those cells to cell death. As more cells and agents become known in the art, the skilled artisan will be able to apply the methods and systems described herein to those as well. Examples of agents include cytokines, interferons, growth factors, chemokines, chemotherapeutics, peptides, polypeptides, nucleic acid sensitizers such as antisense or ribozymes, gene-based sensitizers such as dominant negative gene expression and the like. Other agents include lipids, lipopeptides, sterols and their biosynthetic precursors, polysaccharides, lipopolysaccharides, phosphatase inhibitors, kinase inhibitors, and the like. Further, environmental factors, such as temperature, pH, and the like may be sensitizing agents. Some exemplary sensitizing agents include interferon-γ, interferon-β, phorbol 12-myristate 13-acetate, Bacterodes fragilis enterotoxin, and the like.

With regard to cytokines as sensitizing agents, and in particular, interferon-γ, relevant references include, for example, Koshiji, et al. 1997, “Mechanisms Underlying Apoptosis Induced by Combination of 5-Fluorouracil and Interferon-γ,” Biochemical and Biophysical Research Communications, Article No. RC977657, 240:376-381; Ossina, et al. 1997, “Interferon-γ Modulates a p53-independent Apoptotic Pathway and Apoptosis-related Gene Expression,” Journal of Biological Chemistry, 272(26):16351-16357; Ruiz-Ruiz, et al. 2000, “Interferon-γ Treatment Elevates Caspase-8 Expression and Sensitizes Human Breast Tumor Cells to a Death Receptor-induced Mitochondria-operated Apoptotic Program,” Cancer Research, 60:5673-5680; and Tamura, et al. 1996, “Interferon-γ Induces Ice Gene Expression and Enhances Cellular Susceptibility to Apoptosis in the U937 Leukemia Cell Line,” Biochemical and Biophysical Research Communications, Article No. 1752, 229:21-26.

As used herein, the term cytokine is generic for a diverse group of soluble proteins and/or peptides that act as humoral regulators at nano- to picomolar concentrations and that, under normal or pathological conditions, modulate the functional activities of individual cells and tissues. Such cytokines may also mediate interactions between cells, either directly or indirectly, and may assist in the regulatory activities in the extracellular environment. Thus, it will be understood that growth factors may be cytokines, and that such cytokines may induce, limit or prevent apoptosis, also known as programmed cell death. As will be understood, certain cytokines also behave as classical hormones in that they act at a systemic level, affecting various biological phenomena such as the immune system, inflammation, septic shock, and/or wound healing. Cytokines are generally not produced by specialized cells or in specialized glands. Preferred cytokines include the interleukins, the lympokines, the monokines, the interferons and the chemokines. Other preferred cytokines include the Type-1 cytokines, such as IL2, IFN-γ, IFN-beta, IL12, and TNF-beta (lymphotoxin), and the Type-2 cytokines, such as IL4, IL5, IL6, IL10 and IL13. Preferred cytokines are glycoproteins, and may be produced by, for example, recombinant means or isolated after being secreted by cells.

As used herein, the term apoptosis is intended to mean the physiological process known as programmed cell death, consistent with its accepted meaning as expressed in, for example, U.S. Pat. No. 6,251,614. Thus, apoptosis is intended to refer to a process by which a morphologically and biochemically distinct form of other, non-apoptotic cell death processes that regulate cell turnover under normal physiological conditions. The morphological features of apoptosis include an orchestrated sequence of changes which include cell shrinkage, chromatin condensation, nuclear segmentation and eventual cellular disintegration into discrete membrane-bound apoptotic bodies. The biochemical features of apoptosis include, for example, internucleosomal cleavage of cellular DNA and the activation of ICE/Ced-3 family of proteases. The term apoptosis is used here synonymously with the phrase programmed cell death, but is narrower than the term cell death.

One of skill in the art will readily appreciate that the methods, systems and kits described herein not only apply to compounds that affect cellular growth, including cell death and apoptosis, but also apply to compounds that modulate cellular or biological activity and differentiation. Examples of such activity include antibiotic activity, anti-inflammatory activity, anti-cancer activity, CNS activity, cardiovascular and/or anti-angiogenic activity, renal activity, gastrointestinal activity, uterine activity, anti-parasitic activity, immunomodulatory activity, hematopoietic activity, metabolic activity, for human or veterinary use; insecticidal, and the like. Compounds may be agonists, partial agonists, inverse agonists, reverse agonists, antagonists, competitive antagonists, non-competitive antagonists, and the like.

The cell and agent may be contacted to sensitize the cell. The contacting may be achieved according to any of a number of methods that are well known by those of skill in the art. In one embodiment tumor cells may be cultured according to well known methods and a cytokine, such as IFN-γ, can be added at an appropriate dilution to sensitize or further differentiate the cell. In some instances for example, contacting the cell with the agent may cause the cell to express a protein that can interact with a compound from the set of compounds thereby sensitizing the cell to cell death. As a further example, in other instances, contacting the cell with the agent may cause the cell to express a protein that can interact with a compound from the set of compounds to modulate the growth of the cell, thereby sensitizing the cell. Also, contacting may cause the cell to differentiate some other manner that causes it to interact with the compound from the set of compounds so as to allow identification of compounds that modulate cell growth or further differentiate the cells in some other manner, for example.

In certain embodiments, the agent increases the sensitivity of the cell to cell death by a factor of at least about 2 or 3. In other embodiments the agent increases the sensitivity of the cell to cell death by a factor of at least about 4 or 5. In preferred embodiments sensitivity is increased by a factor of at least about 6, 9, 10, 12, 15, 20, 30, 50, 100, 500, 1000 and the like. Further, the agent may increase the sensitivity of the cell to cell death by a factor as disclosed in the Figures, the Examples, and the accompanying data.

It should be noted that the methods and systems may be used to successively screen and narrow sets of compounds down to one or more compounds. In such situations, sensitivity may be increased by one factor in an initial screen and by a different factor(s) in subsequent screens. For example, in an initial screen, the sensitivity of a cell to death may be increased by a factor of about 10, while in the subsequent screen(s) sensitivity to cell death may be increased by a factor of at least about 3.

The sensitized cell may be contacted with at least one compound from a set or library of compounds. The cell may be contacted with more than one compound, for example a complex mixture or a predetermined number of compounds from a library may be contacted with the sensitized cell. This can allow rapid or high throughput screening of a large number of compounds.

The term set of compounds as used herein, refers to and means broadly, any group, mixture, library, or number of individual chemicals or compounds. In preferred embodiments the set of compounds can be a protein library, a small molecule library, a complex mixture of compounds, such as those derived from and/or extracted from natural sources, already known chemicals that may have unknown uses, and the like. For example, the set of compounds can be a protein library or a combinatorial peptide library. Such libraries are well known in the art and can be generated by well-known methods. For example, a protein library can be obtained by expressing a nucleic acid library. Protein, combinatorial peptide, chemical libraries, and the like, also can be obtained from a variety of commercial sources. The set of compounds also can be a complex compound mixture of any sort that is suitable for the methods described herein. One of skill in the art will appreciate that the set of compounds as described herein is not all-inclusive and that the methods can be applied to any other suitable set of compounds. Individual compounds can be screened one at a time.

The method may also identify a compound or compounds from the set of compounds that modulates the growth of the cell. In some embodiments, initially a group or fraction of compounds can be identified, then additional screening as described (or by any other method) can be done to further characterize an individual compound. In this way, large numbers of compounds or large numbers of complex mixtures can be screened rapidly. Additional embodiments relate to high throughput screening of the set of compounds.

The compound(s) can be identified by detecting some indicia of cellular growth. This can be done by detecting directly or indirectly an indicia of cellular growth of the agent-sensitized cell. Such indicia are well known to those of skill in the art. The indicia of cellular growth may include an indicator of the metabolic activity of the cell, and the like, provided only that these indicia may serve as either direct or indirect indicators of cell growth and/or direct or indirect indicators of the absence of cell death. Examples include whole cell counting using instruments such as Coulter counter (Beckman Instruments); techniques such as viable dye staining and direct counting with a hemacytometer; monitoring DNA synthesis by ³H-thymidine incorporation and radiometric detection, or BrdU incorporation; colorimetric/fluorescent detection with a labeled antibody reactive to BrdU; cell proliferation as monitored by crystal violet cellular staining and calorimetric detection; cell proliferation as monitored by sulforhodamine B cellular staining and calorimetric detection; metabolic activity as monitored by MTT and colorimetric detection; and the like.

Further, in some embodiments, dyes or labels may be used. Any dye or label can be used that is capable of indicating cellular growth or lack thereof. For example, in some embodiments a reduction-oxidation indicator dye may be used. Examples of such dyes include tetrazolium violet, 2-(p-Iodophenyl-3-(p-nitrophenyl)-5-phenyl-tetrazolium chloride, and resazurin. In a preferred embodiment, resazurin is used to detect metabolic activity that corresponds to cellular growth.

In other embodiments, the activity of apoptosis or cell death specific enzymes may be determined. For example, substrate analogs that fluoresce upon cleavage by a particular apoptosis specific enzyme may be used. One such example is the detection of caspase activity, which in some cell systems regulates or participates in the apoptosis pathway. Caspase activity during apoptosis may be determined by measuring the fluorescence of cleaved caspase substrates. Many such enzyme/substrate systems are well known by those of skill in the art.

In another embodiment, the apoptosis associated migration of cellular components can be measured. For example, Annexin V can be used to measure cell viability. Annexin V binds to phosphatidylserine, which is a lipid that is translocated from the its normal location on the inner cell membrane to the outer surface of cell membranes upon cell death. Many other indicators of cell viability or lack thereof are well known in the art.

As mentioned above, the methods and system may be used in a high throughput screen. The methods and systems can rapidly screen sets of compounds, such as, individual compound libraries, compound mixtures, chemical libraries, and the like. Individual compounds from a set of compounds can be screened individually one at a time. Also, groups of compounds or compound mixtures can be screened to quickly narrow a larger number down to a more manageable size. The smaller group can be further screened for individual compounds or mixtures of compounds. One of skill in the art can easily recognize additional variations that are appropriate for a given set of circumstances.

One set of embodiments relates to a high-throughput, tumor cell-based screening method for identifying one or more cell-growth modulating compounds from among a library of compounds. The high-throughput, tumor cell-based screening method can be improved to include the step of contacting the tumor cell to an agent that increases the sensitivity of tumor to cell death.

The tumor cell may be sensitized to cell death by contacting it with a agent, such as cytokine, a small molecule, a chemical, and the like. Any agent described above may be implemented in the contacting step. In a preferred embodiment the agent may be a cytokine, as defined above. For example, the cytokine may be IL2, IFN-γ, IL12, and TNF-beta (lymphotoxin), IL4, IL5, IL6, IL10, IL13, and the like. Those skilled in the art are familiar with many tumor cells and corresponding agents, such as cytokines, that sensitize the tumor cells to death. Any of those tumor cell and corresponding agent, those that may become known, can be used with the high-throughput screening method. The method and system can apply equally to later discovered or characterized tumor cells and corresponding cytokines or agents. Engineered cells and cell lines may also be used. Again, although these embodiments are described in terms of sensitizing a cell to cell death, other embodiments relate to sensitizing cells in order to identify compounds that modulate other cellular activity.

Contacting the agent with the tumor cell can increase the sensitivity of the cell to cell death by some factor, as described above. As an example, in one preferred embodiment, sensitivity may be increased by a factor of at least about 3.

The library of compounds may preferably, but is not necessarily, derived from or obtained from a natural source. The library of compounds may also include synthetically created or semi-synthetically created compounds and/or classes of compounds, and may be produced by combinatorial chemistry techniques known to those of skill in the art. The library of compounds may be any of those as described above in relation to the set of compounds, for example. As another example, the compounds may be derived from or obtained from a natural source, where the natural source may be obtained from a particular biological or environmental source, habitat or micro-habitat such as a terrestrial, a marine, an aquatic, and the like.

Potential sources of compounds include total extracts, fractionated extracts, or pure compounds from 1) prokaryotic micro-organisms (bacteria, archaea), eukaryotic micro-organisms (fungi, algae, protozoans, helminthes), and viruses, viroids, or prions; 2) unicellular (algae) and multicellular plants, 3) vertebrate animals, 4) invertebrate animals. Compounds or compound mixtures may also be from biosynthetic sources such as combinatorially assembled biosynthetic pathways, genetically engineered biosynthetic pathways, or derived by in vitro or in vivo bioenzymatic conversion. Compounds or compound mixtures may also be from chemical synthetic sources such as chemical syntheses, chemical modification, or combinatorial libraries.

The compounds, or libraries of compounds, that may be present in samples from such sources may include compounds having or modulating biological or cellular activity or differentiation. Examples include antibiotic activity, anti-inflammatory activity, anti-cancer activity, CNS activity, cardiovascular and/or anti-angiogenic activity, renal activity, gastrointestinal activity, uterine activity, anti-parasitic activity, immunomodulatory activity, hematopoietic activity, metabolic activity, for human or veterinary use; insecticidal, and the like. Compounds may be agonists, partial agonists, inverse agonists, reverse agonists, antagonists, competitive antagonists, non-competitive antagonists, and the like. A library of such compounds derived or obtained from a natural source may be, and in certain embodiments, is preferably prepared by fractionating of an extract, or numerous extracts, from a natural source.

Further embodiments relate to systems and kits. Kits may include a container for contacting the components of the method. For example, the container may be a single-well or well(s) in a multi-well plate. A 96-well plate may be used, in which case many compounds may be screened on a single plate or set of plates. The container may be a test tube, for example. Those of skill in the art will recognize many other possible containers and plates that may be used for contacting the various materials. The kit may also include any of the other additional reagents and components that are needed to perform the method, including those disclosed in the Examples, for example. The kit may include reagents for culturing a particular type of cell. For example, different eukaryotic cells may require different reagents for proper cell culture. Also, the kit may include the reagents for detecting the indicia of cellular growth and/or the cellular or biological activity. Examples of such indicia have been described herein. In some embodiments the kit may include the sensitizing agent, examples of which have been described herein. It may include the particular cell, examples of which have also been described herein. In some embodiments the user may provide both the cell and the sensitizing agent. It should be noted that the user of the kit may usually provide the set of compounds or the compound library, although this does not have to be the case. One of skill in the art will appreciate the various components that may be included in a kit, consistent with the methods and systems disclosed herein.

All references, patents, articles, and the like, disclosed herein are hereby incorporated by reference in their entirety.

The following examples are intended for illustration purposes only, and should not be construed as limiting the scope of the invention in any way.

### EXAMPLES

### Example 1

A method for screening compounds cytotoxic to HT-29 human colon cell line using IFN-γ sensitized cells, and the results of such a method, are described. The method and the assays described below that incorporate the method, were designed to identify compounds in complex mixtures that affect the growth of human tumor cells. In complex mixtures, the concentration of an individual active compound may be at such a low level that it would not score positive in conventional cytotoxic screens. IFN-γ increases the sensitivity of tumor cell lines to cell death by anti-cancer agents. Sensitization of tumor cells by pretreatment with IFN-γ coupled with a differential screen allows the rapid identification of previously undetected cytotoxic activities in complex mixtures. Also, the screen can be used to identify single compounds that interact with protein targets whose synthesis is induced as a result of pretreatment with IFN-γ. The purified individual active compound can then be used to further evaluate the effectiveness in inhibiting tumor cell growth as well as to determine the mechanism of action.

HT-29 cells (ATCC HTB-38) were cultured in McCoy's 5a Medium with 25 mM HEPES and supplemented with 10% fetal calf serum, 1× nonessential amino acids, 2 mM L-glutamine, 1 mM sodium pyruvate, 100 U/ml penicillin/100 ug/ml streptomycin (complete medium) at 37° C. in a humidified 5% CO₂ cabinet. The cellular monolayer was washed with PBS and adherent cells were removed by treatment with 0.25% typsin/0.05% EDTA. The cells were collected by centrifugation and resuspended in complete medium. Cell number and viability was determined by Trypan Blue exclusion staining.

Duplicate plates of HT-29 cells were prepared by seeding cells into clear bottom, black walled 96 well tissue culture treated microplates (Corning 3904) at 5000 cells/well in a volume of 150 μl per well. The cells were cultured until in exponential phase growth (24-48 hours). 25 μl of recombinant human IFN-γ (R & D Systems, Cat No 285-1F) diluted in complete medium was added to achieve a final concentration of 200 U/ml to one of the duplicate plates and 25 μl of complete medium was added to the remaining plates. The plates were incubated at 37° C. in a humidified 5% CO₂ cabinet for 12-16 hours. Compound extracts prepared from marine microorganisms were diluted in complete medium to 8× their final intended concentration. 25 μl of test extracts was added to the corresponding wells of the duplicate plates at final concentrations ranging from 1-10 μg/ml extract. The final volume in the assay was 200 μl with a final concentration of 0.3-0.5% dimethysulfoxide (DMSO). Untreated cells and control treated cells were included in the screen. The plates were incubated at 37° C. in a humidified 5% CO₂ cabinet for 48 hours. 20 μl of 0.2 mg/ml filter sterilized resazurin (dissolved in PBS) was added to each well and the plates were returned to the incubator for 6-8 hours. Resazurin is an oxidation-reduction dye that detects metabolic activity that corresponds to cellular growth. Reduction of resazurin due to metabolic activity causes the dye to change from a non-fluorescent to fluorescent product that can be measured at 530 nm excitation and 590 nm emission wavelengths. Relative Fluorescent Units (RFU) was measured in a Fluoroskan Ascent microplate fluorometer (Thermo/Labsystems) in a bottom-read mode with excitation at 530 nm and emission at 590 nm. Background subtraction was performed on all wells. Percent growth was defined as (test well RFU-background RFU)/(mean untreated wells RFU-background RFU)×100%. Compounds which showed less than or equal to 50% growth in the presence of IFN-γ pretreatment were scored as positive. Data analysis and graphing was performed with custom Excel (Microsoft) applications and Prism 3.02 (GraftPad Software).

### Example 2

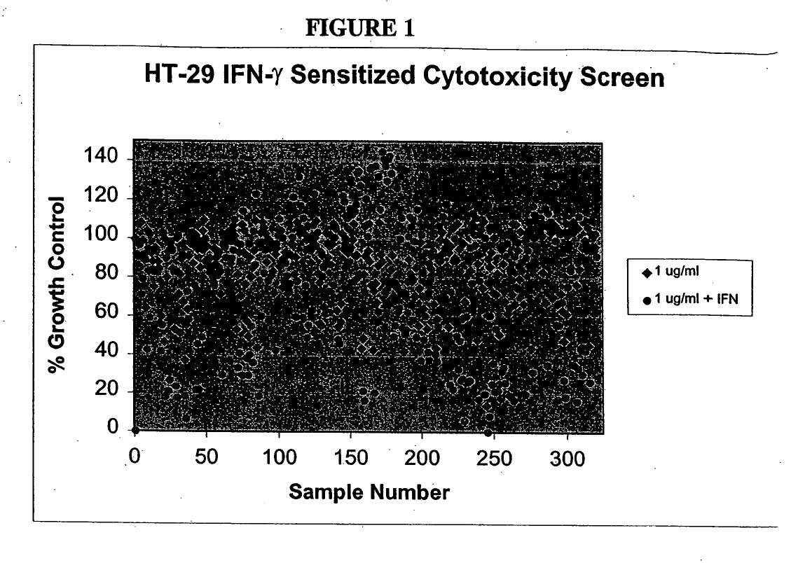

Screens were performed according to the methods described in Example 1. The screens used a threshold of 20% of control growth in absence of compound to define positive active extracts. Only one active extract was identified in the HT-29 screen where the cells were not subject to IFN-γ pretreatment. Using the same criterion, twenty active extracts were identified in the IFN-γ pretreated HT-29 screen. FIG. 1 is a graphic representation of anti-tumor cell activities of 320 natural product extracts screened in the HT-29 cytotoxicity assay in the absence and presence of IFN-γ.

### Example 3

One thousand four hundred forty (1440) crude natural product extracts prepared from cultured marine micro-organisms were screened for their ability to induce cellular death in the IFN-γ sensitized HT-29 screen. The screening was performed using the methods described in Example 1. Extracts that exhibited less than or equal to 20% growth compared to untreated controls in IFN-γ pretreated HT-29 cells, but that exhibited minimal cytotoxic activity in non-pretreated HT-29 cells, were selected for further characterization. Following confirmation of cytotoxicity activity in a full dose response study in HT-29, several strains, including NPS1518, NPS1594, and NPS1386, were chosen for scale up fermentation and preparation of crude extracts.

### Example 4

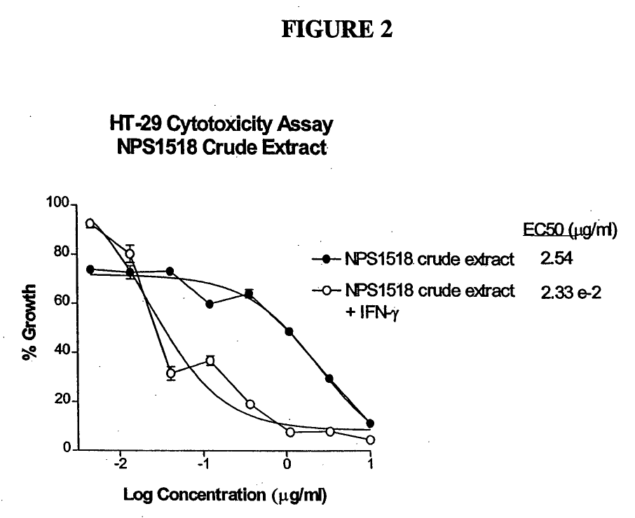

Extracts that scored positive in the primary screen were evaluated for tumor cell death in a dose-dependent cytotoxicity assay. HT-29 cells were untreated or pretreated in triplicate with IFN-γ and incubated with serial dilutions of extract for 48 hours. Inhibition of cellular growth was determined using resazurin indicator dye as described above. As graphically depicted in FIG. 2, the results of this Example indicate that IFN-γ pretreatment increased sensitivity of HT-29 tumor cells to cell death upon exposure to extract NPS1518 in dose-dependent manner. IFN-γ treatment alone had no growth inhibitory effect on HT-29 cells (data not shown). As indicated in FIG. 2, EC50 values for HT-29 cells not subject to IFN-γ pretreatment, according to Example 1, upon exposure to the extract fraction NPS1518, were determined to be 2.54 μg/ml. EC50 values for HT-29 cells subject to IFN-γ pretreatment, upon exposure to NPS1518, were determined to be 2.33×10⁻² ug/ml, thereby yielding an increase in sensitivity upon pretreatment with IFN-γ of approximately 109 times.

### Example 5

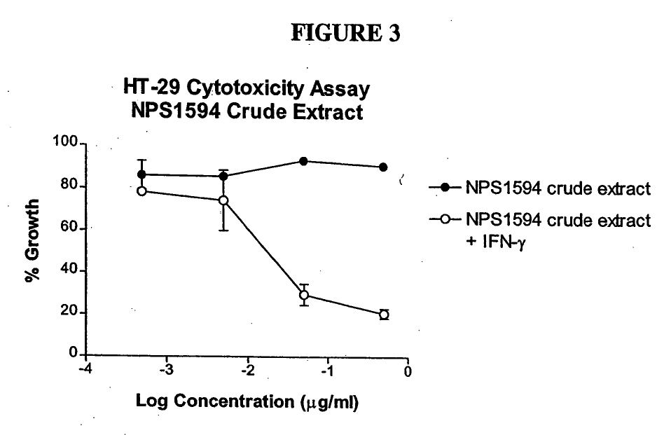

NPS1594, a positive extract identified in the primary screen, also showed enhanced anti-tumor cell activity in IFN-γ presensitized HT-29 tumor cells (FIG. 3). NPS1594 showed significant anti-tumor activity in IFN-γ presensitized cells even at concentrations of crude extract as low as 1 ng/ml.

### Example 6

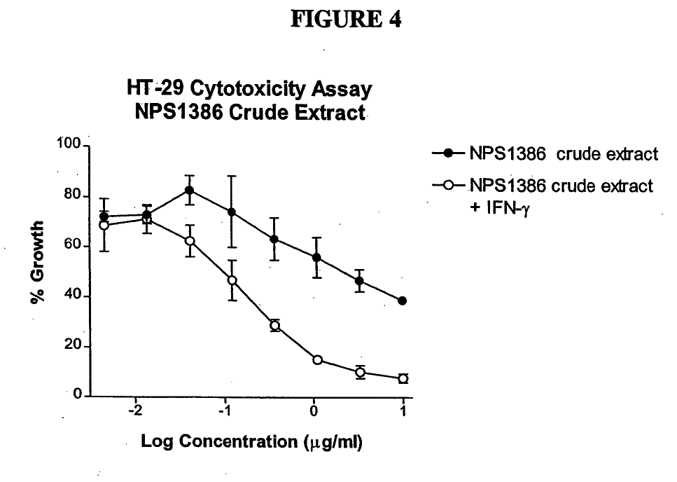

As shown in FIG. 4, HT-29 cells not subject to IFN-γ pretreatment, as described in Example 1, upon exposure to the extract fraction NPS1386, resulted in 50-60% tumor cell death at the two highest concentrations tested. Tumor cell death values for HT-29 cells subject to IFN-γ pretreatment, upon exposure to NPS1386, resulted in 90-100% death. The concentration of NPS1386 extract required to achieve 50% tumor cell death in absence of IFN-γ pretreatment was 2.37 ug/ml. The concentration of extract required to achieve 50% tumor cell death in IFN-γ pretreated tumor cells was 0.1 ug/ml, yielding an increase in sensitivity upon pretreatment with IFN-γ of approximately 24 fold.

### Example 7

10 ul of 1 mg/ml NPS1386 crude extract and partially pure compound samples were analyzed by reverse-phase HPLC (Agilent 1100 series) on a 4.6×75 mm ACE C-18 column (Advanced Chromatography Technologies). The column was equilibrated for 2 minutes at 1 ml/min, 30 C. and samples were eluted with 25-100% gradient of acetonitrile: water. Eluants were monitored by DAD at 420 nm and ELSD.

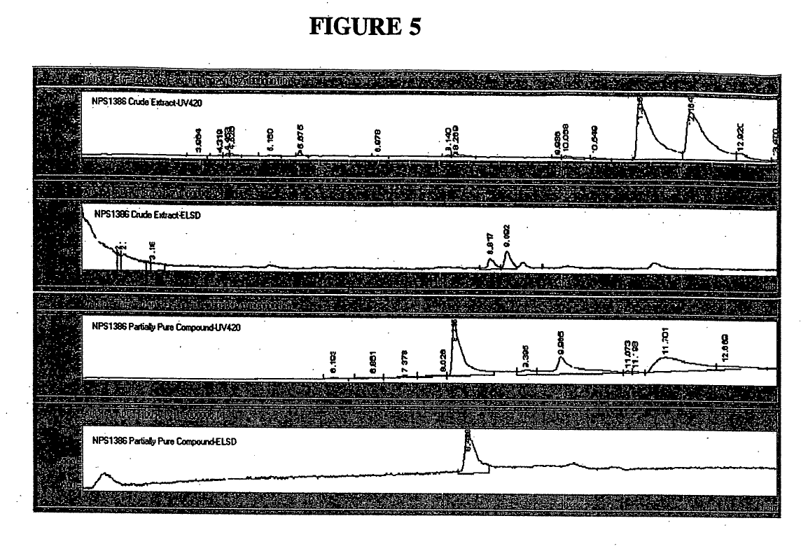

FIG. 5 is a graphic representation of HPLC analyses of NPS1386 crude extract and partially purified NPS1386 compound. The minor peak component in NPS1386 crude extract was barely visible by UV with a retention time of 8.289 minutes. The same minor peak component, however, was not visible by ELSD. NPS1386 activity was isolated using assay-guided fractionation. The partially pure NPS1386 activity was determined to have a major UV adsorbing peak with a retention time of 8.215 minutes corresponding well with the retention time observed in the crude extract. The partially pure NPS1386 activity appeared as a single major component as determined by ELSD with a retention time of 8.428 minutes.

### Example 8

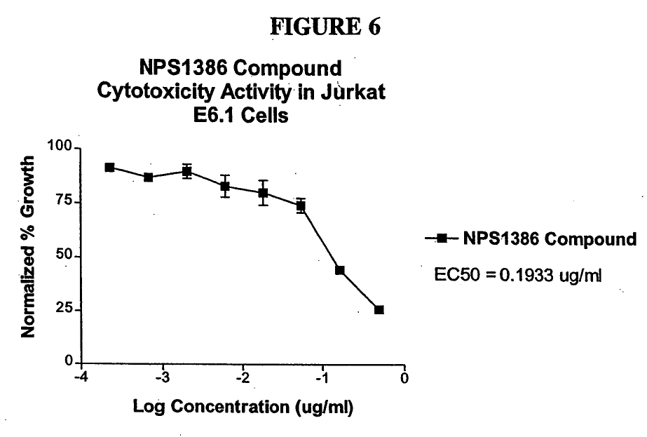

A cytotoxicity assay was performed according to the methods described in Example 1 except Jurkat E6-1 T-cell human leukemia cell line was used. FIG. 6 is a graphic representation of the results of a cytotoxicity assay for Jurkat human tumor cells and partially pure NPS1386 compound. As indicated in the figure, the EC50 value for NPS1386 compound was determined to be 0.193 ug/ml.

### Example 9

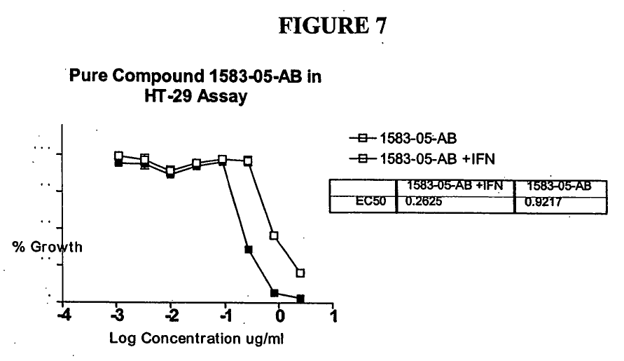

A compound designated 1583-05-AB was discovered according to the methods described herein. The compound was purified from a bacterial crude extract, which showed activity in a primary screen as described above, for example. Human HT-29 cells were seeded in 96 well microtiter plates as described above, and pre-treated with 200 IU human interferon-γ (IFN) after an overnight incubation at 37° C. Duplicate plates of HT-29 cells were pre-treated with a media control after an overnight incubation at 37° C. After 20 hours of treatment with or without IFN, the cells received the compound at the concentrations indicated in FIG. 7. After 48 hours of contact with the compound at the various concentrations, cytotoxicity was determined by the reduction of rezazurin as mentioned above. The concentration of compound that caused a 50% reduction in viability (EC50) was determined by modeling the data using the software package Prizm. As shown in FIG. 7, the use of interferon-γ in this assay in increased the sensitization of the HT-29 cell line approximately 4-fold.

### Example 10

Screens are performed using MCF-7 human breast carcinoma cells and interferon-β. The screens are performed using methods similar to those described in Example 1. The screens use a threshold of 20% of control growth in absence of compound to define positive active extracts. MCF-7 cells are grown in duplicate in 96 well plates. The cells in one plate are pre-treated with interferon-β. The duplicate cells in the other plate (control) have media added to them, but no interferon-β. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using MCF-7 human breast carcinoma cells and interferon-β to determine which have activity individually.

### Example 11

Screens are performed using NIH-OVCAR-3 ovarian carcinoma cells and interferon-β. The screens are performed using methods similar to those described in Example 1. The screens use a threshold of 20% of control growth in absence of compound to define positive active extracts. NIH-OVCAR-3 ovarian carcinoma cells are grown in duplicate in 96 well plates. The cells in one plate are pre-treated with interferon-β. The duplicate cells in the other plate (control) have media added to them, but no interferon-β. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using NIH-OVCAR-3 ovarian carcinoma cells and interferon-β to determine which have activity individually.

### Example 12

Screens are performed using HeLa human ovarian carcinoma cells and interferon-γ. The screens are performed using methods similar to those described in Example 1. The screens use a threshold of 20% of control growth in absence of compound to define positive active extracts. HeLa human ovarian carcinoma cells are grown in duplicate in 96 well plates. The cells in one plate are pre-treated with interferon-γ. The duplicate cells in the other plate (control) have media added to them, but no interferon-γ. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using HeLa human ovarian carcinoma cells and interferon-γ to determine which have activity individually.

### Example 13

Screens are performed using LNCaP human prostate carcinoma cells and phorbol 12-myristate 13-acetate. The screens are performed using methods similar to those described in Example 1. The screens use a threshold of 20% of control growth in absence of compound to define positive active extracts. LNCaP human prostate carcinoma cells are grown in duplicate in 96 well plates. The cells in one plate are pre-treated with phorbol 12-myristate 13-acetate. The duplicate cells in the other plate (control) have media added to them, but no phorbol 12-myristate 13-acetate. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using LNCaP human prostate carcinoma cells and phorbol 12-myristate 13-acetate to determine which have activity individually.

### Example 14

Screens are performed using CaCo-2 human colonic carcinoma cells and Bacterodes fragilis enterotoxin. The screens are performed using methods similar to those described in Example 1. The screens use a threshold of 20% of control growth in absence of compound to define positive active extracts. CaCo-2 human colonic carcinoma cells are grown in duplicate in 96 well plates. The cells in one plate are pre-treated with Bacterodes fragilis enterotoxin. The duplicate cells in the other plate (control) have media added to them, but no Bacterodes fragilis enterotoxin. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using CaCo-2 human colonic carcinoma cells and Bacterodes fragilis enterotoxin to determine which have activity individually.

### Example 15

Screens are performed using HT-29 cells and Bacterodes fragilis enterotoxin. The screens are performed using methods similar to those described in Example 1. The screens use a threshold of 20% of control growth in absence of compound to define positive active extracts. HT-29 cells are grown in duplicate in 96 well plates. The cells in one plate are pre-treated with Bacterodes fragilis enterotoxin. The duplicate cells in the other plate (control) have media added to them, but no Bacterodes fragilis enterotoxin. A library of compositions is screened by adding an aliquot of each composition to one well on both the sensistized and the control plate. Compositions that meet the threshold criteria are identified as positive compositions. The positive compositions are then further characterized in that their individual components are purified or isolated and individually screened using HT-29 cells and Bacterodes fragilis enterotoxin to determine which have activity individually.

What is claimed is:

1. A method of identifying at least one compound, from a set of compounds, that modulates the growth or biological activity of a cell, comprising the steps of:
(a) contacting a cell to an agent that increases the sensitivity of the cell to cell death by at least three-fold, thereby yielding a sensitized or more differentiated cell, wherein the agent does not restore p53 function to the cell;
(b) contacting the sensitized cell to at least one compound from the set of compounds; and
(c) identifying at least one compound that modulates the growth of the cell by detecting an indicia of cellular growth of the sensitized cell.

2. The method of claim 1, wherein the agent is selected from the group consisting of cytokines, interferons, growth factors, chemokines, chemotherapeutics, peptides, polypeptides, nucleic acid sensitizers, gene-based sensitizers, lipids, lipopeptides, sterols and their biosynthetic precursors, polysaccharides, lipopolysaccharides, phosphatase inhibitors, kinase inhibitors, temperature, and pH.

3. The method of claim 2, wherein the cytokine is selected from the group consisting of IL2, IFN-γ, IL12, and TNF-beta (lymphotoxin), IL4, IL5, IL6, IL10 and IL13.

4. The method of claim 1, wherein the agent further increases the sensitivity of the cell to cell death by a factor of at least about 4.

5. The method of claim 1, wherein the agent further increases the sensitivity of the cell to cell death by a factor of at least about 5.

6. The method of claim 1, wherein the agent further increases the sensitivity of the cell to cell death by a factor of at least about 6.

7. The method of claim 1, wherein the agent further increases the sensitivity of the cell to cell death by a factor of at least about 9.

8. The method of claim 1, wherein the agent further increases the sensitivity of the cell to cell death by a factor of at least about 10.

9. The method of claim 1, wherein the agent further increases the sensitivity of the cell to cell death by a factor of at least about 12.

10. The method of claim 1, wherein the agent further increases the sensitivity of the cell to cell death by a factor of at least about 15.

11. The method of claim 1, wherein the agent further increases the sensitivity of the cell to cell death by a factor of at least about 20.

12. The method of claim 1, wherein the agent further increases the sensitivity of the cell to cell death by a factor of at least about 30.

13. The method of claim 1, wherein the agent further increases the sensitivity of the cell to cell death by a factor of at least about 50.

14. The method of claim 1, wherein the agent further increases the sensitivity of the cell to cell death by a factor of at least about 100.

15. The method of claim 1, wherein the agent further increases the sensitivity of the cell to cell death by a factor of at least about 500.

16. The method of claim 1, wherein the agent further increases the sensitivity of the cell to cell death by a factor of at least about 1000.

17. The method of claim 1, wherein the set of compounds is a complex mixture of compounds.

18. The method of claim 1, wherein the method is a high-throughput screen.

19. The method of claim 1, wherein the cell is a tumor cell.

20. The method of claim 19, wherein the cell is selected from the group consisting of HT-29, LoVo, SW620 cell, mesothelioma cell lines, glioma cell lines, ovarian carcinoma cell lines, and human renal cell carcinoma cell lines.

21. The method of claim 1, wherein the cell is infected by an intracellular parasite.

22. The method of claim 21, wherein the intracellular parasite is selected from the group consisting of a virus, a bacterium, a fungus, and a protozoa.

23. The method of claim 1, wherein the indicia of cellular growth is detected by a technique selected from the group consisting of whole cell counting, viable dye staining, direct counting with a hemacytometer, monitoring DNA synthesis by ³H-thymidine incorporation and radiometric detection, BrdU incorporation, colorimetric/fluorescent detection with a labeled antibody reactive to BrdU, cell proliferation as monitored by crystal violet cellular staining and colorimetric detection, cell proliferation as monitored by sulforhodamine B cellular staining and calorimetric detection, and metabolic activity as monitored by MTT and calorimetric detection.

24. The method of claim 1, wherein the indicia of cellular growth is metabolic activity in the sensitized cell.

25. The method of claim 1, wherein the indicia of cellular growth is fluorescence of a dye in the presence of the sensitized cell.

26. The method of claim 25, wherein the dye is selected from the group consisting of tetrazolium violet, 2-(p-Iodophenyl-3-(p-nitrophenyl)-5-phenyl-tetrazolium chloride, and resazurin.

27. The method of claim 1, wherein the biological activity is selected from the group consisting of antibiotic activity, anti-inflammatory activity, anti-cancer activity, CNS activity, cardiovascular and/or anti-angiogenic activity, renal activity, gastrointestinal activity, uterine activity, anti-parasitic activity, immunomodulatory activity, hematopoietic activity, metabolic activity, agonists, partial agonists, inverse agonists, reverse agonists, antagonists, competitive antagonists, and non-competitive antagonists.

28. In a high-throughput, tumor cell-based screening assay method for identifying one or more cell-growth or differentiation modulating compounds from among a library of compounds, the improvement comprising the step of contacting the tumor cells to an agent that increases the sensitivity of the tumor cell to cell death, wherein the agent does not restore p53 function to the cell.

29. The method of claim 28, wherein the agent is selected from the group consisting of cytokines, interferons, growth factors, chemokines, chemotherapeutics, peptides, polypeptides, nucleic acid sensitizers, gene-based sensitizers, lipids, lipopeptides, sterols and their biosynthetic precursors, polysaccharides, lipopolysaccharides, phosphatase inhibitors, kinase inhibitors, temperature, and pH.

30. The method of claim 28, wherein the cytokine is selected from the group consisting of IL2, IFN-γ, IL12, TNF-beta (lymphotoxin), IL4, IL5, IL6, IL10 and IL13.

31. The method of claim 28, wherein the agent increases the sensitivity of the cell to cell death by a factor of at least about 3.

32. The method of claim 28, wherein the agent increases the sensitivity of the cell to cell death by a factor of at least about 6.

33. The method of claim 28, wherein the agent increases the sensitivity of the cell to cell death by a factor of at least about 10.

34. The method of claim 28, wherein the agent increases the sensitivity of the cell to cell death by a factor of at least about 15.

35. The method of claim 28, wherein the agent increases the sensitivity of the cell to cell death by a factor of at least about 30.

36. The method of claim 28, wherein the agent increases the sensitivity of the cell to cell death by a factor of at least about 50.

37. The method of claim 28, wherein the agent increases the sensitivity of the cell to cell death by a factor of at least about 100.

38. The method of claim 28, wherein the agent increases the sensitivity of the cell to cell death by a factor of at least about 500.

39. The method of claim 28, wherein the agent increases the sensitivity of the cell to cell death by a factor of at least about 1000.

40. The method of claim 28, wherein the library of compounds is derived from a natural source.

41. The method of claim 40, wherein the library of compounds is prepared by fractionating an extract.

42. The method of claim 41, wherein the extract is from a natural source.

43. A kit for identifying at least one compound, from a set of compounds, that modulates the growth or biological activity of a cell, comprising a container for contacting a compound or set of compounds with a sensitized cell and at least one additional component selected from the group consisting of a sensitizing agent, a cell, a reagent for detecting an indicia of cellular growth and/or biological or cellular activity, and a growth media.

44. The kit of claim 43, wherein the container is a well in a multi-well container.
